(19) United States
(12) Patent Application Publication
(10) Pub. No.: US 2002/0158244 A1
(43) Pub. Date: Oct. 31, 2002

(54) Method and apparatus for controlling an electric current through bio-molecules

(76) Inventor: Edward William Schlag, Muenchen (DE)

Correspondence Address: KENYON & KENYON, ONE BROADWAY, NEW YORK, NY, 10004, US

(21) Appl. No.: 10/122,662

(22) Filed: Apr. 15, 2002

Related U.S. Application Data
(62) Division of application No. 09/733,458, filed on Dec. 8, 2000.
(60) Provisional application No. 60/170,135, filed on Dec. 10, 1999.

(51) Int. Cl. H01L 35/24; H01L 51/00; C07K 7/00

(52) U.S. Cl. 257/40; 530/324

(57) ABSTRACT

The present invention relates to a method of and an apparatus for controlling an electric current through a medium, comprising the steps of applying a voltage at two electrodes being coupled to the medium at two different positions, and changing of at least one physical parameter of said medium, so as to change the conductivity of said medium.

In order to improve performance of molecular electronic devices it is suggested that said medium comprises a chain molecule (1) including a plurality of molecular sections (2) and changing of said at least one physical parameter of the medium includes one parameter that blocks the internal movement of adjacent molecular sections (2) of said chain molecule (1).

### FIELD OF THE INVENTION

The present invention generally relates to a method of and an apparatus for controlling passage of an electric current through a medium. In particular through a medium comprised of a chain molecule including a plurality of molecular sections, the chain molecule capable of being reversibly changed into and out of a charge conducting state.

### Related Technology

Charge transfer in bio-molecules is subject of increasing interest. This is partially due to the fact that molecular systems may be considered as conductors and switches as described in “Electron transfer from isolated molecules to bio-molecules”, vol. 1 and 2 in Adv. Chem. Phys., vol. 106 and 107 (1999), editors Jortner, J., and Bixon, M.. Bio-molecules may be used as components of molecular circuitries such as logic gates, offering various applications for data processing. Only recently memory devices were built with molecular components, inter alia disclosed in “Electronically configurable molecular-based logic gates” by Collier, C. P., Wong, E. W., Belohradsky, M., Raymo, F. M., Stoddart, U., Kuekes, P. J., Williams, R. S., Heath, J. R., Science 285 (1999), pages 391 - 394. Such memory devices are programmable once and may be used afterwards as programmable read only memories (PROM). Rewriting the memory device, however, is often not possible.

For retrieving data from the molecular memory devices switching elements are necessary. In general, prior art switching elements are silicon-type elements. Thus, while the memory devices are reduced to molecular size, the logic gates for the actual data retrieval and data processing, however, such as AND-gates, OR-gates etc., are realized in prior art technique and are thus comparatively voluminous. Hence, the advantage of molecular memory devices in comparison to silicon-type memory devices as to size and performance, particularly conductivity and heat dissipation, is squandered by silicon-type switching elements.

Another example for devices of molecular electronics is a field effect transistor with a conventional structure, however, with organic materials as substance for parts of the FET. In “Organic-inorganic Hybrid Materials as Semiconducting Channels in Thin-Film Field-Effect Transistors”, Science 286 (1999), pages 945 - 947 such a TFT-structure is disclosed by Kagan, C. R., Mitzi, D. B., Dimitrakopoulos, C. D., on which an organic-inorganic perovskite is deposited as a semiconducting channel, increasing the performance of conventional TFTs. Devices of this type with a conventional structure however are still far beyond molecular dimensions and are thus considerably larger than necessary for the envisaged circuitries.

Moreover, molecular systems with donor, polyene chain and acceptor have been synthesized and electron transfer were initiated therein by light irradiation. However, such systems suffer from various drawbacks upon irradiation with light, such as structural rearrangements in the molecules. This is described in “Physikalische und chemische Grundlagen der Molekularelektronik” by Mahler, G., in Phys. B1.47 (1991), pages 831 -836.

The connection of molecular layers to electric terminals is subject of “Large On-Off Ratios and Negative Differential Resistance in a Molecular Electronic Device”, Chen, J., Reed, M. A., Rawlett, A. M., Tour, J. M., in Science 286 (1999) pages 1550 -1552.

Reed and Tour (Reed and Tour, “Computing with molecules” Scientific American, June 2000) describe a molecular switch which functions by variation of the applied voltage. Further described are the ability of other chemicals to store electrons for up to 10 minutes and twisting of molecule in order to destroy overlapping orbitals leading to a loss of conductivity. The charge transfer of all described molecular devices in that document is based on the conjugated system of overlapping orbitals. Nothing is disclosed in view of a “hopping” charge transfer mechanism.

U.S. Pat. No. 5,589,692 to Reed, which is incorporated herein by reference, discloses electronic systems, wherein microelectronic semiconductor integrated circuit devices are integrated on a common substrate with molecular electronic devices. Reed further describes charge transfer via a hopping mechanism designated as “Coulomb blockade” , wherein the charge transfer structure can be thought of as a series of small capacitors and in which a single electron “hops” through the structure.

### OBJECTS AND SUMMARY OF THE INVENTION

The accepted paradigm for electrical conduction in molecules in the related art is that of a conjugated chain. By contrast, the present invention provides for a new paradigm for electrical conduction in a molecular system exemplified by a polypeptide. Control here means that the process can be externally influenced, i.e. switchable.

Molecular switches can be used for example in controlling of chemical processes. Molecular switches offer the possibilities of transporting a charge over a certain distance and thus the possibility of discharging, activating or controlling a chemical reaction remotely.

The present invention seeks to provide a molecular switch and a corresponding method for controlling charge transfer through a molecule which mitigate or avoid the above disadvantages and limitations of the prior art.

The present invention provides a method of controlling passage of an electric current through a medium comprised of a chain molecule including a plurality of molecular sections, the chain molecule capable of being reversibly changed into and out of a charge conducting state, the method comprising the steps of applying a voltage across the medium and controlling a relative movement of the molecular sections so as to reversibly change the charge conducting state of the chain molecule.

The present invention also provides a molecular switch comprising a chain molecule including a plurality of molecular sections, the chain molecule capable of being reversibly changed into and out of a charge conducting state, and a trigger capable of controlling a relative movement of the molecular sections so as to reversibly change the conductivity of the chain molecule.

Charge transfer from one end of a molecule to the other is a process referred to as “charge hopping”. As described by Baranov, L. Ya., and Schlag, E. W., in “New Mechanism for Facile Charge Transport in Polypeptides”, Z. Naturforsch. 54a(1999) pages 387 - 396, a charge on a polypeptide molecule jumps under certain conditions from one section of the polypeptide molecule to the respective adjacent section of the polypeptide molecule. The sections of the molecule correspond to the amino acids of the polypeptide, respectively. Charge transfer through the polypeptide molecule is between very efficient and completely hampered, depending on the amino acids and their environments. The mechanism is based on hopping of a charge from one amino acid to an adjacent one with its energy being a purely local excitation.

Charge transfer in the polypeptide molecule is the result of a hopping mechanism, and not of an energy band scheme. Transition time of a charge from one amino acid to an adjacent one is in the order of 150 to 200 fsec. This period may be explained by the following model. The carbamide group of each of the amino acids is stiff. It is loosely bound to the adjacent amino acids. In a molecule-based reference system orientation of adjacent amino acids can be represented by two angles (of rotation) ψ and Φ). Adjacent amino acids rotate freely and independently over a large range of ψ and Φ. In a small range, the so-called firing range of ψ and Φ, however, adjacent amino acids reach an isoenergetic state with a small or vanishing energy barrier, leading to an hybrid state. In this position of amino acids the coupling is strong and charge transfer between two adjacent amino acids is easily possible. So if ψ and Φ are beyond the firing range charge transfer is interrupted to at least one side of the charge carrying amino acid; and if ψ and Φ are within the firing range charge transfer is easily accomplished.

A hopping model has been invoked recently to explain charge transport in DNA (Wan C, Femtosecond dynamics of DNA-mediated electron transfer. Proc Natl Acad Sci USA 1999 May 25;96(11):6014-9)

The mechanism which was explained above in connection with polypeptide molecules also applies to the characteristics of other chain bio-molecules with molecular sections pivoting on each other and allowing charge transfer between sections in certain positions only. Because of the hopping mechanism and the related finite transfer time of charges, charge transfer with bio-molecules offers the possibility of selectively controlling a current through the molecular “nano-conductor”. Generally, a new class of molecular electronic devices is achievable using flexible polymer chains (i.e. in particular polypeptides) with a flexibility that allows certain steric configurations resulting from rotation or other relative movements of molecular sections and allowing charge transfer between the molecular sections in the steric configurations. In most of the configurations represented by e.g. critical angles adjacent molecular sections are electronically independent from each other and conductivity is rather poor, however, in some specific configurations a strong coupling occurs between adjacent molecular sections resulting in an efficient conductivity. The different configurations of the molecule are designated as resting state and firing state, respectively. By hindrance of the rotation of the molecular sections in the resting state it is prevented that the molecule reaches its firing state. In other words, controlling the transfer of signals through the molecule is achieved by the prevention of relative movements of molecular sections. The “freezing” of the relative movement of molecular sections of polypeptides is attained by creating an additional binding between adjacent amino acids molecular sections, by a rearrangement of atoms in the molecule, and by further processes, respectively. The binding may be initiated chemically, typically by a redox system, or in a photolytic process. On the other hand the ionization potential of side groups of the molecule can be affected by a photolytic process and may also result in blocking the internal movements of the molecule and thus hinder charge transfer in the molecule. Moreover, even changes of the environment of the molecule may cause a blocking of internal movements in the molecule. An example is electrostriction and initiation of a liquid crystal phase. As a summary it was proven by the inventor that the relation is valid: no movement (within the molecule)—no conduction. Furthermore, the unidirectional flow of current follows local energetic profiles which have properties similar to diodes, an as such, can form components of molecular gates. The electronic state of the molecule can be changed by external means such as the introduction of photons.

The term “chain molecule” refers here to a flexible polymer chain molecule containing molecular sections wherein the polymer chain has a flexibility that allows certain steric configurations resulting from rotation or other relative movements of the molecular sections which allows charge transfer between the molecular sections. Other suited chain molecules are those in which the application of an external electric field induces a dipole moment that leads to electrostriction of the molecule. In general, suited chain molecules are flexible polymer chain molecules that allow a charge transfer via a hopping mechanism. Examples for such polymers comprise “bio-molecules,” such as XX, YY, polypeptides or combinations thereof. Preferably, such chain molecules are polypeptides. The amount of molecular sections of the flexible polymer chain molecule according to the present invention can differ in a wide range from about XX to about XX amino acids, since larger structures do not impede the motion of the chain molecule.

The chain molecules according to the present invention contain a plurality of mutually moving “molecular sections”. In general, these molecular sections are pivoting on each other and allow a charge transfer between sections in certain positions only. They have high internal mobility and isoelectronic states, depending on their configuration. Applicable molecular sections can be XX and/or modified XX, YY and/or modified YY, amino acids and/or modified amino acids or combinations thereof.

In case of a polypeptide, the molecular sections are the singular amino acids. Suited amino acids are those amino acids which can transport a charge via the oxygen of their carbamide group. A similar mechanism would also transport protons and hence produce long-range changes in acidity. Further, amino acids containing a natural chromophore as side group are of particular use to introduce the charge into the system, although redox systems are the natural originators.

In particular, controlling a relative movement of the molecular sections so as to reversibly change the charge conducting state of the chain molecule includes irradiating the medium with electromagnetic radiation of a predetermined wavelength being absorbed by at least one of the plurality of molecular sections. If the invention is applied to a cascade switch in a preferred embodiment, the chain molecule comprises a first molecular section absorbing electromagnetic radiation of a first predetermined wavelength and a second molecular section absorbing electromagnetic radiation of a second predetermined wavelength.

Controlling a relative movement of the molecular sections may include applying an electric field to the chain molecule. The field induces a dipole moment in the molecule and electrostriction of the molecule is caused.

In another embodiment, the medium comprises a layer of chain molecules. This will also enable interchain linkage of charges as another possibility to build circuits by interchain charge transfer. Controlling a relative movement of the molecular sections so as to reversibly change the charge conducting state of the chain molecule comprises in this case applying an external field by a pulsed voltage source so as to cause a fixed orientation of the molecular sections and block charge transfer in the chain molecule.

For controlling a chemical process with light, the molecular switch can comprise a chromophore as a donor and an acceptor, both as a contact electrode of the molecule. The radiation source for electromagnetic radiation emits accordingly electromagnetic radiation of a predetermined wavelength being absorbed by the chromophore so as to release an electrical charge from the chromophore into the chain molecule.

Alternatively, controlling a relative movement of the molecular sections can include changing at least one physical parameter of the medium, for example, by using a pulsed voltage source for applying an electric field to the chain molecule so as to induce a dipole moment and to cause an electrostriction process of the molecule.

In another alternative embodiment the medium comprises a layer of chain molecules. The means for changing the at least one physical parameter of the medium comprises a pulsed voltage source or an electrochemical potential. Changing of the parameter includes, in this case, creating and applying an external field to the molecule so as to block the orientation of the molecular sections fixedly and to prevent charge transfer in the chain molecule.

In general, for a controllable electric conductor, a chain molecule may be used, which chain molecule comprises molecular sections pivoting on each other with a period of rotation smaller than the smallest period of vibration of the molecule. In particular the chain molecule is a polypeptide molecule and the molecular sections are amino acids.

One advantage of the invention is its high efficiency compared to typical silicon- based electronic circuits and its low cost implementation. This offers a vast field of applications to molecular switches.

### BRIEF DESCRIPTION OF THE DRAWINGS

The invention will become clearer from the following description of embodiments, by way of example only, with reference to the accompanying drawings, in which:

FIG. 1 illustrates the principal arrangement of a first embodiment of the apparatus according to the invention;

FIG. 2A and 2B illustrate paths of the angles of rotation ψ and Φ of amino acid(s) in the phase space, respectively; and

FIG. 3 illustrates schematically another embodiment of the apparatus according to the invention.

### DETAILED DESCRIPTION

In the following the invention will be described according to a first embodiment wherein controlling of charge transfer includes irradiation of a molecule with electromagnetic radiation. The medium comprises one or a plurality of polypeptide molecules as the chain molecules each with a plurality of amino acids as molecular sections.

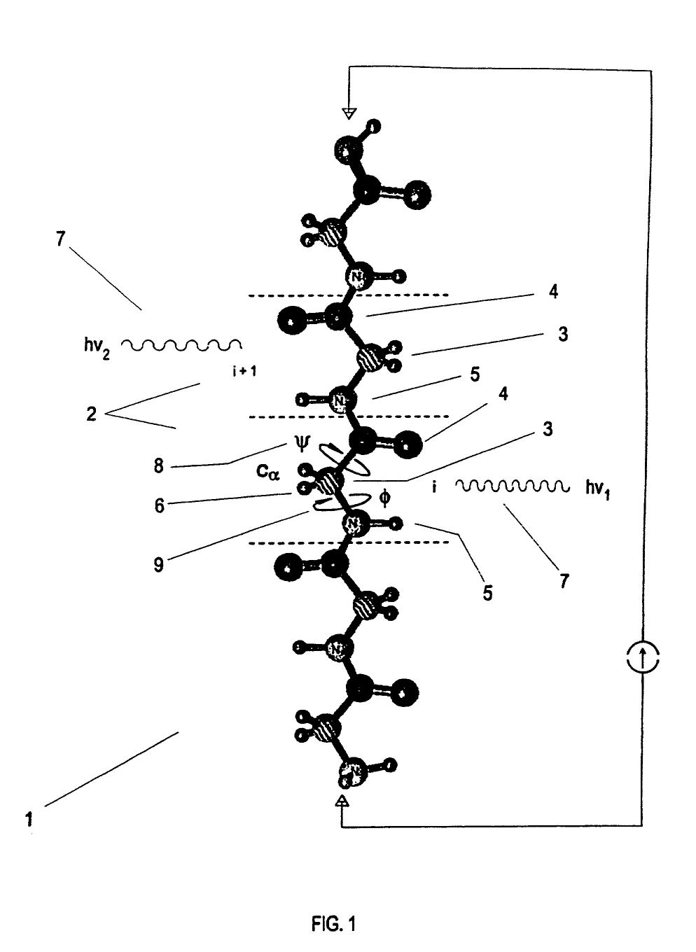

FIG. 1 illustrates a polypeptide molecule 1 comprising a chain of a plurality of amino acids 2. The amino acids are Glycin designated with an index i and i+1, respectively. A central C_{α}-atom 3 of Glycin is referred to as C_{α}-atom in the following. The C_{α}-atom 3 is a hinge for a CO-group 4 and a NH-group 5 in the molecule.

As explained above the CO-group 4 rotates in relation to the C_{α}-atom 3 about a first angle of rotation 8, referred to as ψ. Correspondingly, the NH-group 5 rotates about a second angle of rotation 9, referred to as Φ. At a predetermined value of Φ and ψ oxygen atoms of two adjacent amino acids 2 come close so as to form a hybrid state. In this configuration a charge may easily jump from amino acid i to the adjacent amino acid i+I, if there is a charge on an amino acid i. However, the charge remains on the “input” side of the C_{α}-atom 3 as long as Φᵢ₋₁and ψ_{i,i+1} have not reached a critical angle allowing the charge to jump to the next section i+1 of the polypeptide molecule. Only in this critical position of the amino acids 2, that is in the critical range of Φ and ψ, the potential surface between neighbor amino acids becomes isoenergetic. With the isoenergetic potential surface the charge is free to move in the direction which is determined by an external field. The external field is illustrated by the two contact electrodes above and below the polypeptide molecule in FIG. 1. The contact electrodes are coupled to a (DC) current source.

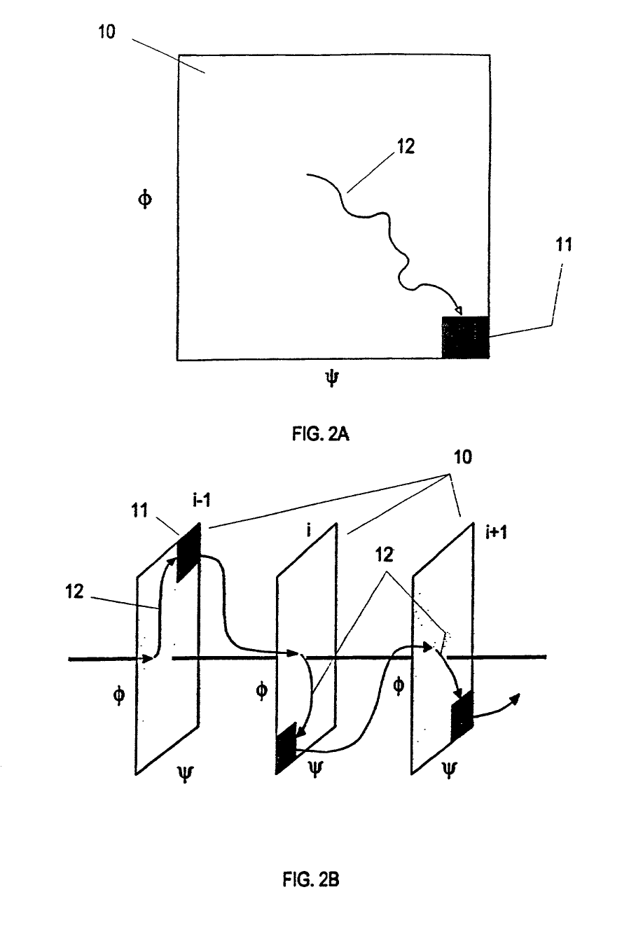

The angles Φ and ψ show a stochastic behavior, not a coherent behavior. This is illustrated in the corresponding Ramachandran plots 10 in FIG. 2A and 2B. In FIG. 2A a possible trajectory 12 is shown in the two-dimensional phase space 10 of a system with the co-ordinates Φ and ψ. As soon as both angles 8 and 9, that is Φ and ψ enter simultaneously a “window” or switching range 11, depicted as a shaded square in FIG. 2, the precondition for the charge to jump from amino acid 2 to neighbor amino acid 2 is fulfilled: the charge leaves section i of the polypeptide molecule I and jumps to section i+1 of polypeptide molecule 1.

In FIG. 2B the movement of the charge over several amino acids 2 is schematically illustrated. The charge moves from left to right, each of the Ramachandran-plots 10 corresponds to respective amino acids 2 of the polypeptide molecule 1. Each of the Ramachandran-plots comprises the small switching range 11. Within the first plot 10 on the left hand side of FIG. 2B the critical angles Φ and ψ move along a trajectory 12, which reaches the switching range 11 after a certain period of time allowing the charge to enter the next plot, i.e. the plot in the middle of FIG. 2B. In the second plot the critical angles Φ and ψ have arbitrary start values within the plane 10 and move along a second trajectory 12, reaching the switching range 11 of the second plot 10 after a certain period of time. Then, the charge jumps from the plot 10 in the middle of FIG. 2B to the plot 10 on the right hand side of FIG. 2B, etc.

The transition rate of the charge between neighboring amino acids i and i+1 depends on the period of time for Φ and ψ to reach the switching range 12. The average period of time for Φ and ψ to reach range 12 is in the order of 170 fsec. In other words, charge transfer is on one hand side fast enough not to be disturbed by relaxation such as vibrational relaxation etc. and it is on the other hand side slow enough to offer the possibility of interrupting/suspending charge transfer.

In order to be able to control the rotation of the CO-group 4 and of the NH-group 5 around C_{α}-atom 3 and to control both co-ordinates Φ and ψ specifically (i.e. to disturb them) so as to hinder charge transfer, the molecule 1 is irradiated with electromagnetic radiation 7. This is illustrated in FIG. 1. The electromagnetic radiation 7 has an energy hν and a corresponding wavelength λ which is absorbed by at least one of the amino acids 2. Such a radiation absorption is easier to achieve with other amino acids than Glycin. For example one of H-atoms 6 of Glycin at the C_{α}-atom 3 may be replaced by an arbitrary R-group (not shown). This results in a different amino acid 2 as a component of polypeptide 1. The energy hν may be absorbed by the R-group (not shown) of the molecule. Thus the state of the R-group is changed and rotation of the CO-group 4 and NH-group 5 around C_{α}-atom 3 is not free anymore. Since therefore the respective oxygen-atoms of neighboring amino acids 2 do not come as close any more as before charge transfer between amino acids 2 is hampered now: irradiation of the polypeptide molecule inhibits conduction, the molecule is in a “blocking state”. Only upon ending irradiation of the polypeptide molecule 1 free rotation of the CO-group 4 and the NH-group 5 is enabled again and thus charge transfer may take place: the polypeptide molecule 1 is in a “conductive state”.

This characteristic may be used to “freeze” a charge on an amino acid—at least for a short period of time up to some picoseconds. Namely, when charge transfer is inhibited on both sides of an amino acid or a group of amino acids the charge is “captured” in this portion of the polypeptide molecule. This effect of a “captured” charge may be exploited for “buffering” a charge. Only upon ending the irradiation the charge is released again and may proceed to one end of the molecule.

Due to the explained switching characteristics of polypeptide molecules I various application examples for molecular switches are conceivable, two of which will be discussed below.

First application example:

The switching characteristics of the polypeptide molecule 1 is similar to the one of a switching transistor and offers a simple realization of an AND-gate of which input and output are listed in table 1 below.

TABLE 1

| injection | switching state | output |
|---|---|---|
| 0 | 0 | 0 |
| 0 | 1 | 0 |
| 1 | 0 | 0 |
| 1 | 1 | 1 |

The first column “injection” of table 1 contains the respective state of an amino acid 2 of the molecule 1. If there is a charge at the respective amino acid i then the first column contains 1, if there is no charge at the respective amino acid i then the first column contains 0. In the second column of the truth table the switching state of the molecule 1 is listed. If the molecule 1 is e.g. irradiated the rotation of amino acid i is hindered, the conductivity of the molecule is substantially zero. This state is designated 0 in the second column. In the reverse case of free rotation of the amino acid i the molecule is “conductive”. This state is designated 1 in the second column. Only with 1 in column “injection” and simultaneously in column “switching state” there is a 1 in column “output” of the truth table, i.e. the charge is passed on from amino acid i of polypeptide molecule 1 to the adjacent amino acid i+1. Thus table I corresponds to a truth table of an AND-gate.

“Switching” of the molecule is carried out by electromagnetic irradiation of the side groups in the molecule in a first embodiment of the invention. As explained above the electromagnetic excitation results in a change of the electronic state of at least one of the side groups with a first type of molecules as medium. By the change of the electronic state the rotation of the respective groups is hampered and charge transfer from one molecular section to another is prevented with high efficiency. With a second type of molecules as medium electromagnetic excitation leads to “bridging” within the molecule or to other steric interferences between neighboring molecular sections and thus again to preventing neighboring molecular sections from free rotation and from exchanging charges.

In this first preferred embodiment with the molecule being “switched” by electromagnetic excitation a single molecule or a plurality of molecules are arranged on a supporting substrate (not shown). In particular the substrate may comprise a semiconductor substrate. At both ends of the polypeptide molecule 1 a respective contact electrode is provided for. The radiation source for irradiation of the polypeptide molecule 1 is preferably a semiconductor laser (not shown) on the same substrate as the molecular switch. Preferably a plurality of molecular switches are arranged on the substrate all of them being irradiated by the same semiconductor laser. In this arrangement complex logic gates may be realized on a single chip, being irradiated by a common light source.

The above AND-gate that is controlled by electromagnetic excitation can readily be extended. Instead of a single amino acid a plurality of amino acids may be employed within a single polypeptide molecule 1, each of the amino acids absorbing radiation of a different wavelength. As explained above this can be achieved by substituting different R-groups at the respective C_{α}-atoms 3. If such polypeptide molecule 1 with different amino acids 3 is irradiated with two energies hν₁, and hν₂ charge transfer is interrupted at two sites in the molecule 1. Moreover, if a charge is located on molecular section(s) in between the two sites of charge transfer interruption the charge is “captured”. As soon as one of the energies is turned off the respective transition between adjacent amino acids is open for charges again. Thus cascades of AND-gates may be realized wherein portions of the polypeptide molecule 1 may be specifically switched by a predetermined wavelength.

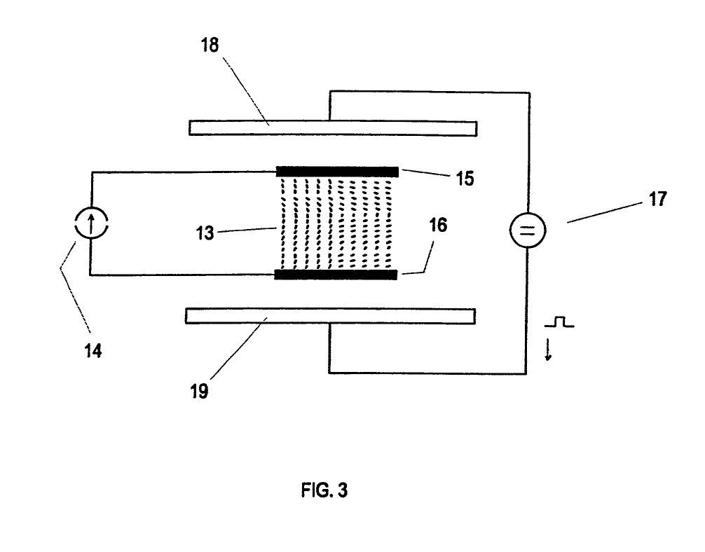

In a second preferred embodiment “switching” of the molecule includes applying an external electric field so as to induce a dipole moment in the chain molecule and thus creating mechanical changes in the molecule (electrostriction). Charge transfer within the molecule is hampered by electrostriction. Controlling the molecule by an external electric field offers also the possibility of employing layers of chain molecules as medium and thus of switching larger currents. In this embodiment the field is applied to the layer of chain molecules rendering molecular sections immobile and thus hindering charge transfer. The according apparatus is illustrated in FIG. 3.

The medium 13 is a layer in the embodiment of FIG. 3. A current through the medium that is to be switched is delivered from a current source 14 via a first contact electrode 15 and a second contact electrode 16. For this embodiment the polypeptide molecules 1 are deposited as an aligned layer (analogous, but not identical to, Langmuir-Blodgett-layers) on a supporting surface thus creating a layer of aligned molecules. The supporting substrate is preferably made of metal, e.g. of gold or silver. Simultaneously, the supporting substrate is used as a contact electrode 15 and 16, respectively, for the layer. The second contact layer 16 and 15, respectively, may be vapor deposited on the medium layer 13. In FIG. 3 the molecules of this layer are schematically shown as columns of dashes. The dashes correspond to molecular sections. As soon as the sections of a molecule are substantially parallel to each other in this symbolic representation (e.g. column on the left most side in FIG. 3) the molecule is “switched”, i.e. conductive. In the case of molecular sections being substantially diagonal (column on the right most side in FIG. 3) the molecule is in a blocking state. However, with the above explanation of the principle of “charge hopping” it will be understood that it is not necessary to have all of the molecular sections in parallel or diagonal, but only the adjacent sections which are about to exchange a charge.

In order to prevent transition of a charge from one molecular section to another molecular section small field pulses of about 0,2V are applied to the molecular layer 13 at a predetermined time. Thus the apparatus of FIG. 3 comprises a pulsed voltage source 17 being coupled to two field electrodes 18 and 19. The field electrodes 17 and 18 are arranged with respect to the medium such that a dipole moment is induced in the layer 13 resulting in a orientation of the molecular sections in the molecules which hampers charge transfer.

An electric potential for hampering transition of an electric charge from one molecular section to another may be attained by application of an external field via field electrodes as explained above but may also be attained by excitation of a chromophore group of the chain molecule. In this case the electromagnetic excitation of the chromophore changes the redox potential at a specific site within the molecule I thus hampering the rotation of molecular sections and preventing the exchange of charges between neighboring amino acids.

Second application example:

The apparatus according to the invention may also be used for triggering and controlling chemical reactions. To that order a chromophore is provided at one end of the polypeptide molecule 1 that is excited by a laser source. Due to the electronic excitation of the chromophore a charge is released from the chromophore that migrates along the molecule 1 in a direction that is determined by any external field. This charge migration from the chromophore over the polypeptide to an acceptor at the other end of the polypeptide 1 may be interrupted by irradiating the molecule with electromagnetic radiation that is absorbed by at least one of the plurality of amino acids 2 of the polypeptide 1. The acceptor at the polypeptide I may in turn discharge a chemical reaction of a coupled molecule. However, the respective amino acids 2 inhibits “hopping” of the charge to a downstream amino acid upon absorption of radiation. Only upon ending the irradiation the charge is free again to move on to the acceptor. This situation corresponds to an AND-gate for two optical input signals and an electrical output signal. The truth table of this AND-gate corresponds to table 1 with the interpretation of releasing a charge as “injection” and absorption of electromagnetic radiation as “switching” the molecule. Due to this possibility of controlling chemical processes by the method according to the invention it is the first time that chemistry can be “controlled from remote”.

In general, for controllable electric conductors chain molecules may be employed having mutually moving molecular sections with a period of rotation which is smaller than the smallest period of vibration of the molecule. For controlling charge transfer or current through a bio-molecule or a layer of bio-molecules the principle of rotation controlled charge mobility is exploited, which was discovered by the inventor. The period of rotation of two adjacent molecular sections of the chain molecule is preferably below 1 picosecond, which ensures a sufficient distance from the smallest period of vibration in the chain molecule. This precondition is satisfied by polypeptides as chain molecules and amino acids as molecular sections.

While the invention has been described in terms of switching electric currents, those of skill in the art will understand based on the description of charge transfer herein that it is not limited merely to such examples but is applicable also to photosynthesis such as in chlorophyll or to solar energy circuitries such as with typical amorphous silicon layers and that the full scope of the invention is properly determined by the claims that follow.

1. A method of controlling passage of an electric current through a medium comprised of a chain molecule including a plurality of molecular sections, the chain molecule capable of being reversibly changed into and out of a charge conducting state, the method comprising the steps of:
applying a voltage across the medium; and
controlling a relative movement of adjacent molecular sections so as to reversibly change the charge conducting state of the chain molecule.

2. The method as recited in claim 1 wherein the controlling of the relative movement of the molecular sections includes irradiating the medium with electromagnetic radiation, the electromagnetic radiation being absorbed by at least one of the plurality of molecular sections.

3. The method as recited in claim 2 wherein the electromagnetic radiation for irradiating the medium comprises at least a first predetermined wavelength and a second predetermined wavelength, and wherein the chain molecule comprises a first molecular section absorbing electromagnetic radiation of the first predetermined wavelength and a second molecular section absorbing electromagnetic radiation of the second predetermined wavelength.

4. The method as recited in claim 1 wherein the controlling a relative movement of the molecular sections comprises applying an electric field to the chain molecule.

5. The method according to claim 1 wherein the controlling a relative movement of the molecular sections comprises applying an external field by a pulsed voltage source so as to cause a fixed orientation of the molecular sections and block charge transfer in the chain molecule.

6. The method as recited in claim 1 wherein the chain molecule is a polypeptide and the molecular sections are amino acids.

7. A molecular switch comprising:
a chain molecule including a plurality of molecular sections, the chain molecule capable of being reversibly changed into and out of a charge conducting state; and
a trigger capable of controlling a relative movement of adjacent molecular sections so as to reversibly change the conductivity of the chain molecule.

8. The molecular switch as recited in claim 7 wherein the relative movement includes a relative rotation of the molecular sections.

9. The molecular switch as recited in claim 7 further comprising:
a donor and an acceptor associated with the chain molecule so as to allow charge to flow from the donor to the acceptor when the chain molecule is in a charge conducting state.

10. The molecular switch as recited in claim 7 wherein the chain molecule is a polypeptide and the molecular sections are amino acids.

11. The molecular switch as recited in claim 9 wherein the chain molecule is a polypeptide and the donor and acceptor are amino acids.

12. The molecular switch as recited in claim 7 further comprising a chromophore associated with the chain molecule so as to allow charge to flow through the chain molecule when the chain molecule is in a charge conducting state.

13. The molecular switch as recited in claim 7 wherein the trigger compris es a radiation source for electromagnetic radiation.

14. The molecular switch as recited in claim 13 wherein the radiation source is capable of emitting electromagnetic radiation of a predetermined wavelength for absorption by the molecular sections.

15. The molecular switch as recited in claim 7 wherein the trigger comprises a pulsed voltage source for applying an electric field to the chain molecule.

16. The molecular switch as recited in claim 7 further comprising a second chain molecule.

17. The molecular switch as recited in claim 7 wherein the trigger comprises a pulsed voltage source for creating an external field so as to fixedly block orientation of the molecular sections.

18. The molecular switch as recited in claim 7 wherein the molecular switch is used in at least one of a logic circuit and a logic gate.

19. The molecular switch as recited in claim 7 wherein the trigger comprises a redox agent.

20. Use of a chain molecule as a molecular switch, wherein the chain molecule comprises molecular sections pivoting on each other with a period of rotation smaller than the smallest period of vibration of the chain molecule.
